(19) United States
(12) Patent Application Publication
(10) Pub. No.: US 2005/0222672 A1
(43) Pub. Date: Oct. 6, 2005

(54) Ostial stent

(75) Inventor: Ascher Shmulewitz, Tel Aviv (IL)

Correspondence Address: KUDIRKA & JOBSE, LLP, ONE STATE STREET, SUITE 800, BOSTON, MA, 02109, US

(73) Assignee: Cappella, Inc., Westford, MA (US)

(21) Appl. No.: 11/095,983

(22) Filed: Mar. 31, 2005

Related U.S. Application Data
(60) Provisional application No. 60/558,312, filed on Apr. 1, 2004.

(51) Int. Cl. A61F 2/06

(52) U.S. Cl. 623/1.15

(57) ABSTRACT

An intraluminal device for placement in a side-branch vessel associated with a main vessel via an ostial region. The intraluminal device includes a stem portion and a toroidal cap portion. The stem portion of the device is placed in the side branch vessel and the toroidal cap portion is placed adjacent to the ostial region.

### RELATED APPLICATIONS

This application claims priority from provisional patent application Ser. No. 60/558,312 filed Apr. 1, 2004 and titled “DEVICE FOR MEDICATION-DISPENSING AND/OR SELECTIVE BLOCKING OF A PREDETERMINED REGION,” the entire subject matter and contents of which are incorporated herein by reference for all purposes.

### FIELD OF THE INVENTION

The invention relates to intraluminal devices for treatment at ostial regions of a vessel.

### BACKGROUND OF THE INVENTION

In today's society, many people suffer from a buildup of a plaque layer covering one or more segments of a coronary vessel where the lesion obstructs the flow of blood through the vessel. This buildup is referred to as a coronary lesion. Often, this condition is treated by placing medical devices or appliances within a patient for supporting the blood vessels or other lumens within the body that have been re-enlarged following cardio balloon angioplasty.

With regard to angioplasty, typically an endovascular or intraluminal implant known as a stent is placed within the blood vessel. A stent is usually tubular in shape and may have a lattice or connected-wire tubular construction. The stent is usually placed within the vessel in a compressed state and then allowed to expand. The support structure of the stent is designed to prevent early collapse of a vessel that has been weakened and damaged by angioplasty. The support provided by the stent prevents the vessel from either closing, referred to as restonosis, or suffering spasms shortly after the angioplasty procedure, and has been shown to facilitate the healing of the damaged vessel wall, a process that occurs over a number of months. Self-expanding and balloon-expandable stents are well known.

During the healing process, inflammation caused by angioplasty and stent implant injury often causes smooth muscle cell proliferation and regrowth inside the stent, thus partially closing the flow channel, i.e., restenosis, thereby reducing or eliminating the beneficial effect of the angioplasty/stenting procedure. Blood clots may also form inside of the newly implanted stent due to the thrombotic nature of the stent surfaces, even when biocompatible materials are used to form the stent.

While large blood clots may not form during the angioplasty procedure itself, or immediately after the procedure, due to the current practice of injecting powerful anti-platelet drugs into the blood circulation, some thrombosis is always present, at least on a microscopic level on stent surfaces. This microscopic thrombosis is thought to play a significant role in the early stages of restenosis by establishing a biocompatible matrix on the surfaces of the stent whereupon smooth muscle cells may subsequently attach and multiply.

Stent coatings are known which contain bioactive agents that are designed to reduce or eliminate thrombosis or restenosis. Such bioactive agents may be dispersed or dissolved in either a bio-durable or bio-erodable polymer matrix that is attached to the surface of the stent wires prior to implant. After implantation, the bioactive agent diffuses out of the polymer matrix and into the surrounding tissue over a period lasting at least four weeks, and in some cases up to one year or longer, ideally matching the time course of restenosis, smooth muscle cell proliferation, thrombosis or a combination thereof.

Some coronary lesions may develop in coronary bifurcations, i.e., a bifurcated vessel including a main vessel associated via an ostial region with a side-branch vessel. Bifurcation lesions may be categorized according to the location of the lesion in the bifurcated vessel. In one example, a type 4a bifurcation lesion may refer to a lesion on the wall of the main vessel in proximity to the ostial region.

Treating bifurcation lesions, e.g., type 4a lesions, using the conventional methods described above, may result in at least part of the plaque layer “drifting” into the side-branch. This effect, commonly referred to as “the snow-plow effect,” may lead to a partial blockage of the side-branch, which may be treated by deploying one or more additional stents into the bifurcated vessel.

Conventional methods for treating bifurcation lesions may include deploying a first stent part in the main branch covering the side branch, and then inflating a “kissing balloon” and deploying a second stent part in the side branch, thereby to form a “T-stent” structure. Such methods as these, however, may result in the T-stent disrupting/obstructing the blood flow from the main vessel to the side branch.

Other stenting methods and/or specially designed bifurcation stents, for example, the Jostent® B stent, the Invatec Bifurcation stent, or the AST stent, may be relatively bulky and may have limited tractability, limited maneuverability and limited access to small caliber vessels.

### SUMMARY OF THE INVENTION

In accordance with one embodiment of the present invention, an intraluminal stent includes a stem portion, defining a lumen therethrough, having a proximal end and a distal end and oriented along a central axis, the stem portion having proximal and distal openings. A cap portion is located at the distal end of the stem portion and comprises a plurality of cap sections oriented substantially orthogonal to the central axis and positioned about the distal opening.

In accordance with another embodiment of the present invention, the intralumenal stent is formed by providing a substantially linear piece of material; and forming a cylindrical stem portion, defining a lumen therethrough, oriented along a central axis, where the cylindrical stem portion has a proximal end and a distal end and proximal and distal openings, respectively. A cap portion is formed at the distal end of the cylindrical stem portion, the cap portion comprising a plurality of cap sections oriented substantially orthogonal to the central axis and positioned about the distal opening.

In another embodiment of the present invention, a method for placing an intraluminal stent includes: providing an intraluminal stent having a stem portion and a cap portion; and positioning the intraluminal stent in a patient such that the stem portion is in a side branch vessel portion and the cap portion extends into a main vessel from the side branch vessel portion. In addition, the cap portion is compressed against an ostial region of the main vessel adjacent the side branch vessel portion.

### BRIEF DESCRIPTION OF THE DRAWINGS

The above and further advantages of the invention may be better understood by referring to the following description in conjunction with the accompanying drawings in which:

FIGS. 1A-1C are isometric-view illustrations of an intraluminal device according to an exemplary embodiment of the invention;

FIG. 2 is a schematic illustration of a target tissue proximal to an ostial region of a bifurcated vessel;

FIG. 3 is a schematic illustration of a catheter tip arrangement according to exemplary embodiments of the invention;

FIG. 4 is a flowchart of a method of inserting an intraluminal device into a bifurcated vessel according to exemplary embodiments of the invention;

FIGS. 5a-5d are schematic illustrations of exemplary stages of inserting an intraluminal device into a bifurcated vessel in accordance with the method of FIG. 4;

FIG. 6 is a schematic illustration of an intraluminal device according to another exemplary embodiment of the invention; and

FIGS. 7a and 7b are schematic illustrations of a closed state and an expanded state, respectively, of an intraluminal device according to yet another exemplary embodiment of the invention.

It will be appreciated that for simplicity and clarity of illustration, elements shown in the drawings have not necessarily been drawn accurately or to scale. For example, the dimensions of some of the elements may be exaggerated relative to other elements for clarity or several physical components included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of the invention. It will be understood by those of ordinary skill in the art that the present invention may be practiced without these specific details. In other instances, well-known methods, procedures, components and structures may not have been described in detail so as not to obscure the present invention.

Embodiments of the invention may include an intraluminal device configured to selectively block at least part of a predetermined region, e.g., an ostial region, of a bifurcated vessel and/or to dispense medication substantially uniformly across at least part of the predetermined region, as described below.

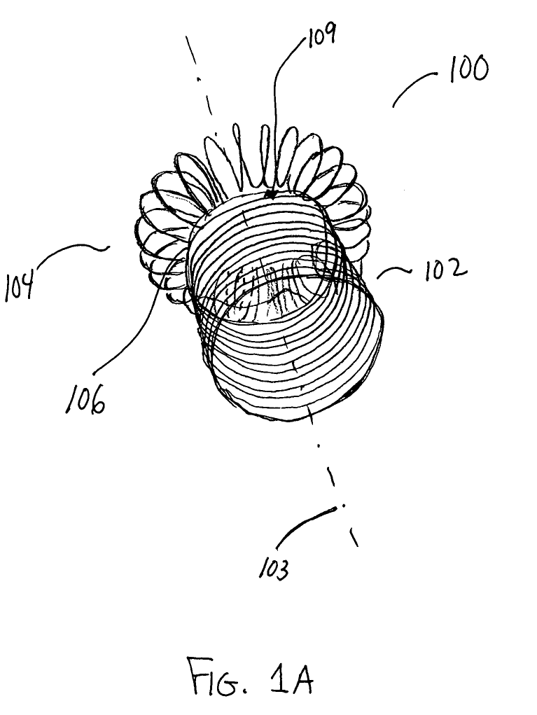

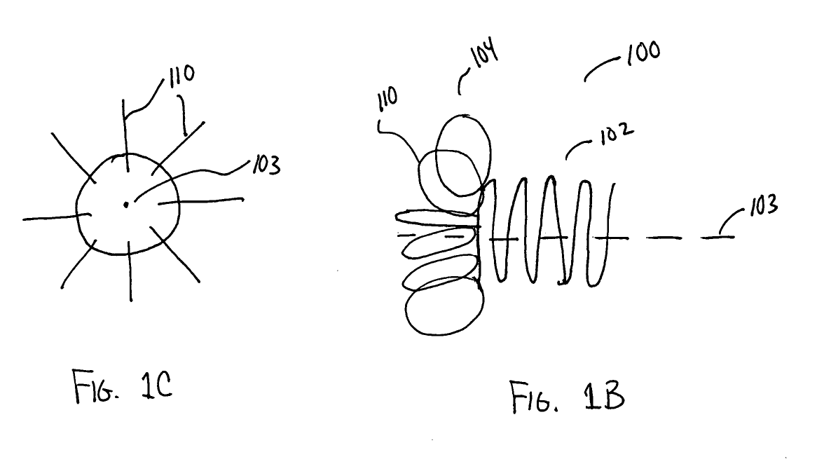

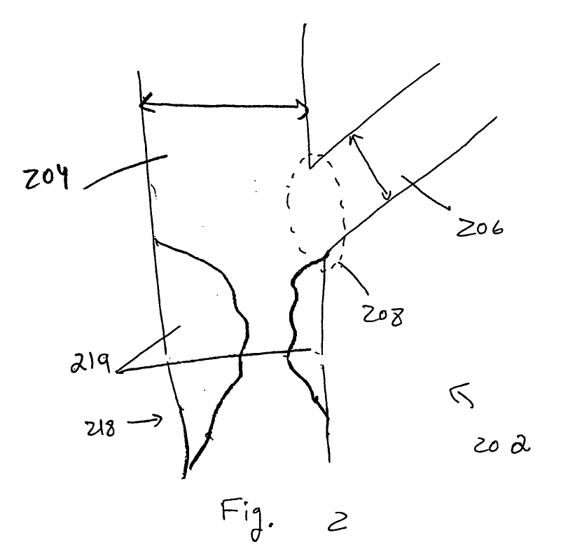

Reference is made to FIGS. 1A and 1B, which illustrates isometric views of an intraluminal device 100 according to an exemplary embodiment of the invention, and to FIG. 2, which schematically illustrates a bifurcated vessel 202 including a main vessel 204 and a side branch vessel 206 extending from the main vessel 204.

The bifurcated vessel 202 may include a target tissue, for example, a diseased segment (a “lesion”), which may include a plaque layer 219 obstructing the flow of blood through the diseased segment of the vessel. The lesion may be located along at least part of the main vessel 204, the side branch vessel 206 and/or an ostial region 208 between the side-branch vessel 206 and the main vessel 204. For example, a type 4a bifurcation lesion 218 may be located in the main vessel 204 in proximity to the ostial region 208.

According to exemplary embodiments of the invention, the intraluminal device 100 may be deployed in the side-branch vessel 206, e.g., as described below, before applying an angioplasty device, e.g., a stent or a balloon as are known in the art, for example, for treating the lesion 218. The intraluminal device 100 may be configured to protect the ostial region 208 and/or the side branch vessel 206 by selectively blocking at least part of the ostial region 208 in order, for example, to prevent the plaque layer 219 or parts thereof from migrating into the side branch vessel 206 by the snow-plow effect, which may result from applying the angioplasty device, as described below.

According to exemplary embodiments of the invention, as shown in FIGS. 1A and 1B, the intraluminal device 100 may include a stem portion 102 configured to fit into the side-branch vessel 206. The intraluminal device 100 may also include a cap portion 104 associated with a first end 106 of the stem portion 102, and configured to selectively block at least part of the ostial region 208, as described in detail below.

According to exemplary embodiments of the invention, the stem portion 102 may have a generally tubular, e.g., spring-like, structure, which may be circularly symmetric with respect to a central axis 103. An outer diameter of the stem portion 102 may be compatible with, i.e., approximately equal to or slightly larger than, an inner diameter of the side branch vessel 206. According to some exemplary embodiments of the invention, the outer diameter of the stem portion 102 may be substantially constant along the central axis 103. According to other embodiments, the outer diameter of the stem portion 102 may vary along the central axis 103, e.g., in order to enable an improved positioning and/or “anchoring” of the stem portion 102 with respect to the side branch 206 and/or to ease the insertion of the intraluminal device 100 into the side branch. For example, the stem portion 102 may have a generally conical shape, i.e., the outer diameter of the stem portion 102 may monotonically increase or decrease along the central axis 103.

According to exemplary embodiments of the invention, the cap portion 104 may have a generally annular shaped, e.g., coiled or toroidal, structure. The cap portion 104 includes a plurality of hoops or rings 110 positioned about the first end 106 of the intraluminal device 100. The rings 110 are oriented substantially orthogonal to the central axis 103 as viewed from the cap portion 104 looking into the stem portion 102, as shown in FIG. 1C. The number of rings 110 is chosen based on the particular anatomy in which the intraluminal device 100 is to be placed. An inner diameter of the cap portion 104 perpendicular to the central axis 103 may be approximately equal to the inner diameter of the stem portion 102, and an outer diameter of the cap portion 104 perpendicular to the central axis 103 may be larger than the outer diameter of the stem portion 102.

According to exemplary embodiments of the invention, the intraluminal device 100 may be formed of a generally elastic, in-vivo stable and/or “shape-memorizing” material, i.e., a material able to be initially formed in a desired shape, e.g., during an initial procedure performed at relatively high temperature, to be deformed, e.g., compressed, and to assume the desired shape in which it was previously shaped. The intraluminal device 100 may be formed of a Nickel-Titanium alloy (“nitinol”) wire. The wire may have a diameter of between 0.003 inches and 0.01 inches, for example, 0.004 inches. A first part of the wire may be coiled to form the predetermined tubular shape of the stem portion 102. A second part of the wire may be bent in relation to the stem portion 102 at a predetermined angle, i.e., of ninety degrees, and may be coiled to form the predetermined annular shape of the cap portion 104. The angle between the cap portion 104 and the stem portion 102 may be predetermined, e.g., based on a specific shape and/or dimensions of the bifurcated vessel 202. As one non-limiting example, the angle may be based, inter alia, on an angle between the main vessel 204 and the side branch 206 and/or a difference between the diameters of the main vessel 204 and the side branch 206.

The device 100 may be formed from a single piece of material or may be assembled in sections. In an alternate embodiment, the cap portion 104 may be of a different material than the stem portion 102. The cap portion 104 may be formed from any compliant material known to one of ordinary skill in the art, e.g., a polymeric material. Further, the cap portion 104 may be formed from a non-compliant material.

According to exemplary embodiments of the invention, the intraluminal device 100 may be configured such that the cap portion 104 may be at least partially compressed when subject to a predetermined pressure, e.g., six atmospheres or more, applied to the cap portion 104, e.g., by an angioplasty device as described below, in a direction generally parallel to the central axis 103. The width of the wire forming the intraluminal device 100, the material of which the wire is formed, and/or the shape and/or the size of the cap portion 104 may be predetermined according to a desired degree of compression of the cap portion 104 and/or the magnitude of the pressure to be applied to the cap portion 104.

According to exemplary embodiments of the invention, at least part of the intraluminal device 100 may be coated with a layer of a desired medication or a material having desired properties to carry and subsequently apply and/or dispense a desired medication. The stem portion 102 and/or the cap portion 104 may be coated with a controlled-release polymer and/or drug, as known in the art, for reducing the probability of undesired side effects, e.g., restenosis. The restenosis may occur as a result of a percutaneous procedure performed on the bifurcated vessel 202, e.g., including insertion of an angioplasty device into the bifurcated vessel 202.

According to exemplary embodiments of the invention, the spacing between neighboring coils of the cap portion 104 and/or the stem portion 102 may be predetermined based on any desired criterion. The spacing between neighboring coils of the cap portion 104 may be predetermined based on a desired dosage and/or distribution uniformity of the medication. In some embodiments, the smaller the spacing between the coils of the cap portion 104, the higher the dosage of the medication that may be applied to the ostial region 208 and the higher the degree of uniformity in which the medication is distributed. Additionally or alternatively, the spacing between neighboring coils of the cap portion 104 may be predetermined based on the shape, size and/or texture of a plaque layer, e.g., of the lesion 218, which may be present in the main vessel 204 and/or the ostial region 208. The spacing between neighboring coils of the cap portion 104 may be sufficiently small to prevent the “snow-plow effect” in the vicinity of the ostial region 208, e.g., to reduce or prevent migration of the plaque layer 219 or parts thereof into the main vessel 204, as a result of the percutaneous procedure described above.

The intraluminal device 100 may not always be visible to a physician viewing, for example, an X-ray fluoroscopy device while deploying and/or positioning the intraluminal device 100 into the bifurcated vessel. According to some exemplary embodiments of the invention, at least one marker 109 may be attached to the intraluminal device 100 at one or more predetermined locations. The marker 109 may be formed of platinum or any other relatively heavy metal, which may be generally visible by X-ray fluoroscopy. The marker 109 may be attached, for example, to the intraluminal device 109 approximately at the first end 106 to allow a relatively high degree of accuracy for positioning the intraluminal device 100 into the bifurcated vessel 202, e.g., by aligning the marker 109 with the ostial region 208.

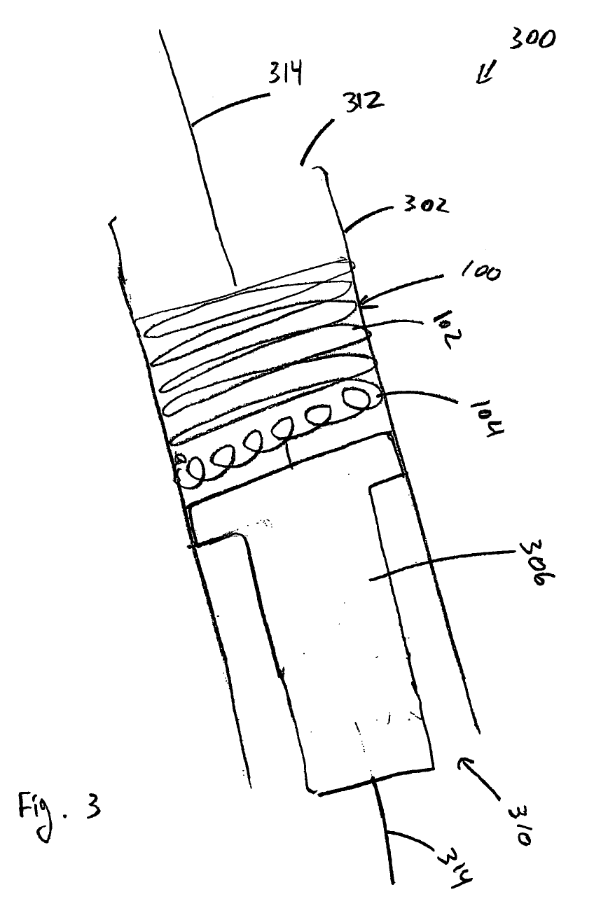

Reference is also made to FIG. 3, which schematically illustrates a catheter tip arrangement 300 for deploying and/or positioning the intraluminal device 100 in a predetermined vessel, e.g., the bifurcated vessel 202, according to exemplary embodiments of the invention.

According to exemplary embodiments of the invention, the catheter tip arrangement 300 may be installed in a catheter tip 302, e.g., as is known in the art. The intraluminal device 100 may be inserted into the catheter tip 302 through an opening in a proximal end 310 of the catheter tip 302. The cap portion 104 may be inserted into the catheter tip 302 after first inserting the stem portion 102. This may be done after compressing the cap portion 104 and/or the stem portion 102 to a size suitable for insertion into the catheter tip 302. For example, the cap portion 104 may be compressed in a direction substantially perpendicular to the central axis 103 (FIG. 1). The catheter tip arrangement 300 may also include a pushing rod 306 configured to coaxially fit into the catheter tip 302 and designed, when activated by the physician, to push the intraluminal device 100 out of a distal end 312 of the catheter tip 302, as is known in the art. The catheter tip arrangement 300 may also include a guide wire 314, as is known in the art.

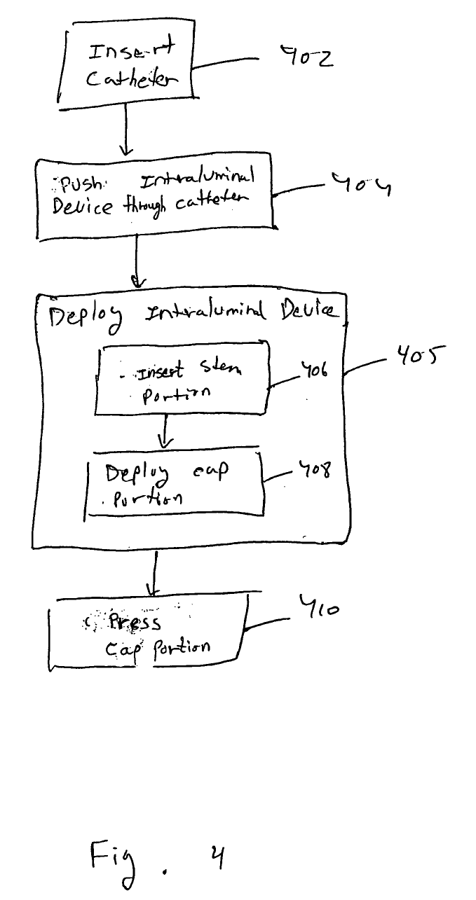

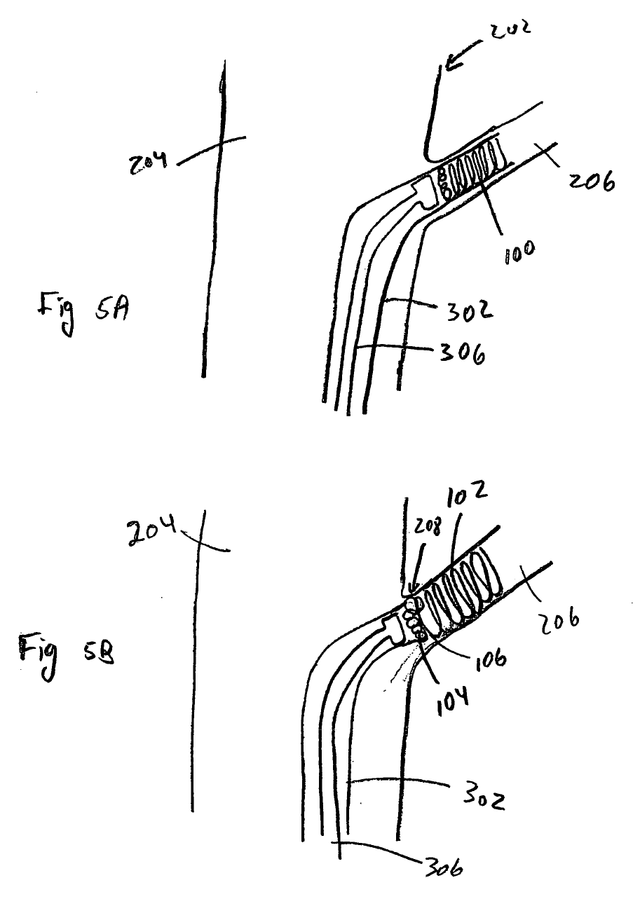

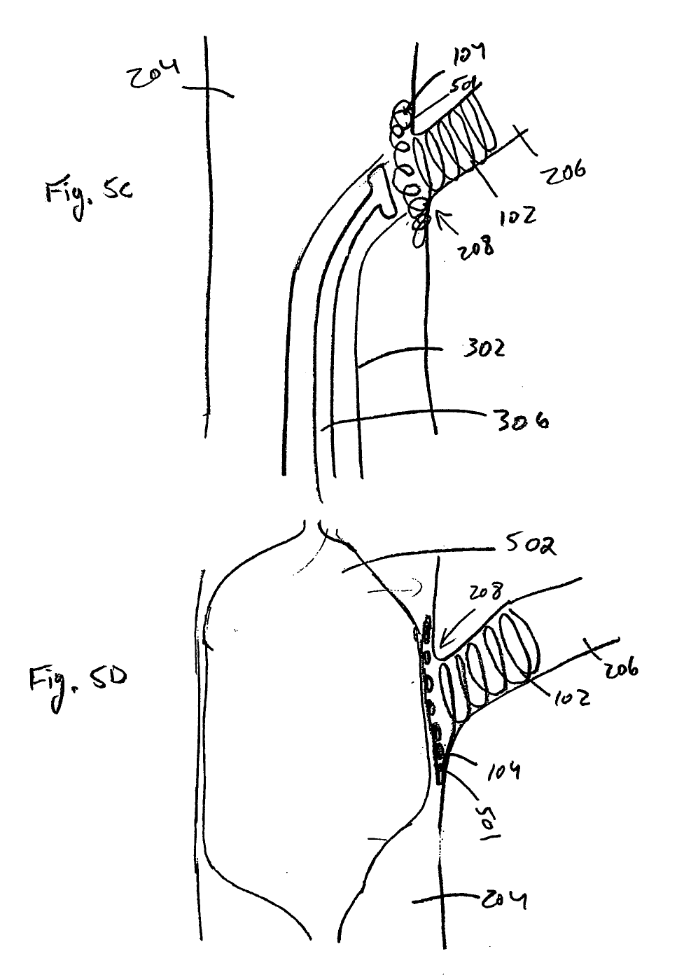

Reference is now made to FIG. 4, which schematically illustrates a flowchart of a method of inserting the intraluminal device 100 into a bifurcated vessel 200 according to exemplary embodiments of the invention, and to FIGS. 5a-5d, which schematically illustrate exemplary stages of inserting the intraluminal device 100 into the bifurcated vessel 200 in accordance with the method of FIG. 4.

As indicated at block 402, the method may include inserting a catheter into a blood vessel and guiding the catheter into the side-branch vessel 206. Any suitable insertion and guidance method, as is known in the art, may be used for inserting and guiding the catheter into the side-branch vessel 206. The catheter may be inserted through, and guided from, one of the femoral arteries by a physician using a monitor to display the position of the catheter in a patient's body, as is known in the art.

As indicated at block 404, the method may also include pushing the intraluminal device 100 through the catheter, e.g., using the pushing rod 306. The physician may activate an appropriate control for moving the pushing rod 306.

As indicated at block 405, the method may further include deploying and positioning the intraluminal device 100 in the bifurcated vessel portion 202, e.g., as described below.

As indicated at block 406, deploying and positioning the intraluminal device 100 may include inserting the stem portion 102 of the intraluminal device 100 into the side-branch vessel 206. This may be achieved by pushing the intraluminal device 100, using pushing rod 306 and/or by pulling back the catheter tip 302. Upon being released from the confines of the catheter tip 302, the deployed stem portion 102 may then expand in the side branch vessel 206. As shown in FIG. 5b, the stem portion 102 may be positioned such that a perimeter of the stem portion 102 is generally in contact with the surface of the side branch vessel 206 and the first end 106 of the stem portion 102 is located at a desired position relative to the ostial region 208.

As indicated at block 408, deploying and positioning the intraluminal device 100 may also include deploying and positioning the cap portion 104 of the intraluminal device 100 in the ostial region 208. This may be achieved, for example, by pushing the intraluminal device 100 using the pushing rod 306 and/or by pulling back the catheter tip 302. The deployed cap portion 104 may then expand in the ostial region 208. As shown in FIG. 5c, the cap portion 104 may be expanded in the main vessel 204 and may be in contact with an ostial surface 501 of the main vessel 204.

As indicated at block 410, the method may further include pressing the deployed cap portion 104 against the ostial surface portion 501. This may be achieved by using an angioplasty device, e.g., a balloon 502 and/or a stent as are known in the art. The angioplasty device may be deployed into the main vessel 204 using any suitable method as is known in the art. The angioplasty device may then be expanded, by inflating the balloon 502, as is known in the art. As shown in FIG. 5d, the balloon 502 may be inflated such that cap portion 104 is pressed between balloon 502 and the ostial surface 501. As a result of this pressure, the cap portion 104 may be at least partly compressed, in a direction generally parallel to the central axis 103 (FIG. 1), and the shape and size of the cap portion 104 may change. The degree of pressure applied to the cap portion 104 and/or a final angle between the cap portion 104 and the stem portion 102 may be related, for example, to the diameter of the side branch vessel 206. When compressed, the cap portion 104 may protect the ostial region 208 and/or the side branch vessel 206, e.g., by selectively blocking migrating tissue from entering the axial region 208, to prevent plaque migration from the main vessel 204 to the side branch vessel 206, as described above. Additionally or alternatively, the medication coating on the cap portion 104 may be substantially uniformly dispensed across the ostial region 208, as described above.

It will be appreciated by those skilled in the art, that the cap portion 104, when being pressed by the angioplasty device, may cause substantially no damage to the bifurcated vessel 202, because the cap portion 104 is formed of a suitable malleable material.

It will be appreciated by those skilled in the art that although some exemplary embodiments of the invention may include a device formed of a wire, e.g., as described above, other embodiments of the invention may include an intraluminal device 100 having any suitable shape and/or size and formed of any suitable material, e.g., as described below, able to selectively block the ostial region 208 and/or dispense a medication substantially uniformly across the ostial region 208.

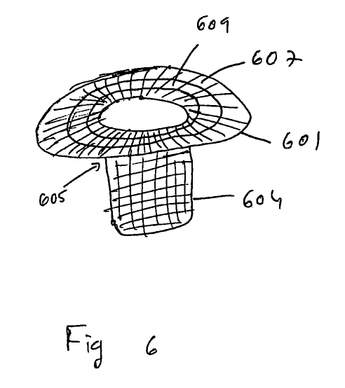

Reference is now made to FIG. 6, which schematically illustrates a “top-hat” type intraluminal device 600 according to another exemplary embodiment of the invention.

The “top-hat intraluminal device 600 may be formed of a wire mesh of any suitable “shape-memorizing” material, e.g., nitinol. The wire forming the wire mesh may have a diameter of, for example, between 0.002 inches and 0.005 inches. The top-hat intraluminal device 600 may include a cylindrical stem portion 604 configured to fit into the side branch vessel 206 of the bifurcated vessel portion 202, and a disk-shaped portion 601 configured to engage the ostial region 208 between the side branch vessel 206 and the main vessel 204, e.g., in analogy to the above description referring to FIGS. 1 and 2. The diameter of the disk-shaped portion 601 may be larger than the diameter of the cylindrical stem portion 604. For example, the diameter of the disk-shaped portion 601 may be between 10% and 50% larger than the diameter of the cylindrical stem portion 604.

According to some exemplary embodiments of the invention, edges of disk-shaped portion 601 may be relatively flexible to conform to the three-dimensional surface topography of the bifurcated vessel portion 202. For example, the wire mesh forming the disk-shaped portion 601 may include “unwoven” edges and/or “cells” of different size, e.g., outer cells 607 that are closer to the outer diameter of the disk-shaped portion 601 may be larger than inner cells 609 that are closer to the center of the disk-shaped portion 601. Accordingly, the outer diameter of the disk-shaped portion 601 may be able to conform to the three-dimensional surface topography of the main vessel 204, the ostial region 208, the angle between the side branch and the main surface, and/or the topology of the bifurcated vessel surface.

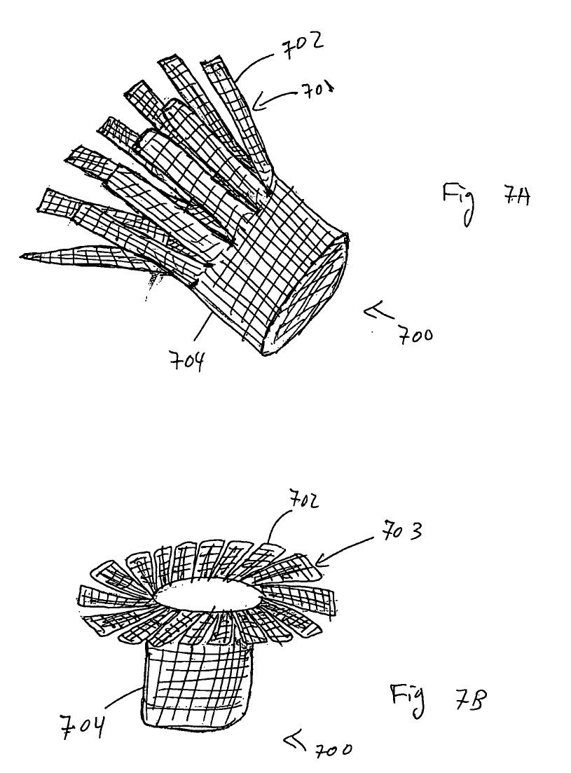

Reference is now made to FIGS. 7A and 7B, which schematically illustrate a closed state and an expanded state, respectively, of a feathered intraluminal device 700 according to yet another exemplary embodiment of the invention.

The feathered intraluminal device 700 may include a cylindrical stem portion 704 configured to fit into a side branch vessel 206 of a bifurcated vessel portion 202, and a cap portion 701 configured to engage the ostial region 208 between the side branch vessel 206 and the main vessel 204, e.g., in analogy to the above description referring to FIGS. 1 and 2. The cylindrical stem portion 701 may include a plurality of elongated elements 701 which may define a generally disk shaped structure 703 when the feathered intraluminal device 700 is expanded as shown in FIG. 7B. The size of the elongated elements 701 may be predetermined, for example, based on the shape and/or size of the side branch vessel 206, the ostial region 208 and/or the main vessel 204. The length of the elongated elements 701 may be predetermined such that the diameter of disk shaped surface 703 may be larger than the diameter of portion 704.

According to exemplary embodiments of the invention, the feathered intraluminal device 700 may be deployed in the bifurcated vessel portion 202 using a balloon configured to urge the cylindrical stem portion 704 into the side branch vessel 206 and to press the elongated element 701 against a surface of the main vessel 204, when the balloon is inflated, to cause the elongated elements 701 to “fold” by deformation from their unexpanded position in FIG. 7A to their expanded disk-shaped position shown in FIG. 7B.

Although some embodiments of the invention described above may refer to an intraluminal device configured for capping a bifurcated coronary vessel and for dispensing medication, it will be appreciated by those skilled in the art that the intraluminal device according to other embodiments of the invention may be configured for capping any other bifurcated lumen, artery or vessel, e.g., in the vascular, biliary, genitourinary, gastrointestinal and respiratory systems, which may have narrowed, weakened, distorted, or otherwise deformed, and/or for dispensing any other substance across at least part of the lumen, artery or vessel, e.g., the carotid artery or trachea bifurcations.

The medicinal coating can include, e.g., and not meant to be limiting, any one or more of the following: paclitaxol, rapamyacin, and heparin.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1. An intraluminal stent comprising:
a stem portion, defining a lumen therethrough along a central axis, having a proximal end and a distal end and having proximal and distal openings, respectively; and
a cap portion, located at the distal end of the stem portion, the cap portion comprising a plurality of cap sections oriented substantially orthogonal to the central axis and positioned about the distal opening.

2. The stent of claim 1, wherein each of the stem portion and the cap portion is formed from a material having shape memory characteristics.

3. The stent of claim 1, wherein the cap portion is capable of being permanently deformed upon application of a force in a direction substantially parallel to the central axis.

4. The stent of claim 1, wherein each cap section of the cap portion has a circular cross-section.

5. The stent of claim 1, wherein an outer diameter of the stem portion is substantially unchanged along a length from the proximal end to the distal end.

6. The stent of claim 1, wherein an outer diameter of the stem portion varies along a length from the proximal end to the distal end.

7. The stent of claim 6 wherein the outer diameter varies monotonically from the proximal end to the distal end.

8. The stent of claim 1, wherein the stem portion comprises a substantially tubular structure.

9. The stent of claim 8, wherein the stem portion comprises a spring-like structure.

10. The stent of claim 9, wherein the cap portion comprises a substantially toroidal shape.

11. The stent of claim 10, further comprising:
a medicinal coating disposed on at least one of the cap portion and the stem portion.

12. The stent of claim 10, wherein the stem portion and the cap portion are formed from a material having shape memory characteristics.

13. The stent of claim 12, wherein the stem portion and the cap portion are formed from a single continuous piece of material.

14. A method of forming an intralumenal stent, the method comprising:
provided a substantially linear piece of material;
forming, from the material, a cylindrical stem portion, defining a lumen therethrough oriented along a central axis, the cylindrical stem portion having a proximal end and a distal end and proximal and distal openings, respectively; and
forming a cap portion at the distal end of the cylindrical stem portion, the cap portion comprising a plurality of cap sections oriented substantially orthogonal to the central axis and positioned about the distal opening.

15. The method of claim 14, further comprising:
choosing the material to be one having shape-memory characteristics.

16. The method of claim 14, further comprising:
coating at least one of the stem portion and the cap portion with a medicinal coating.

17. The method of claim 14, further comprising:
forming the cap portion as a toroid.

18. The method of claim 14, further comprising:
forming the stem portion to have a substantially constant outer diameter.

19. The method of claim 14, further comprising:
forming the stem portion to have an outer diameter that varies along a length from the proximal end to the distal end.

20. The method of claim 19, wherein the outer diameter varies monotonically from the proximal end to the distal end.

21. The method of claim 14, further comprising:
forming each cap section to have a circular cross-section.

22. The method of claim 21, further comprising:
forming the stem portion as a spring.

23. A method comprising:
providing an intraluminal stent having a stem portion and a cap portion;
positioning the intraluminal stent in a patient such that the stem portion is in a side branch vessel portion and the cap portion extends into a main vessel from the side branch vessel portion; and
compressing the cap portion against an ostial region of the main vessel adjacent the side branch vessel portion.

24. The method of claim 23, further comprising:
inflating a balloon catheter in the main vessel to compress the cap portion.

25. The method of claim 23, wherein positioning the stem portion in the side branch vessel comprises:
positioning the stem portion in the side branch vessel portion via an approach from the main vessel.

26. The method of claim 25, wherein positioning the stem portion in the side branch vessel further comprises:
releasing the stent from a delivery catheter, wherein the stem portion is released prior to the cap portion.

27. The method of claim 23, wherein the cap portion comprises:
a plurality of cap sections oriented substantially orthogonal to a central axis of the stem portion and positioned about a diameter of the stem portion.

28. The method of claim 27, wherein:
each cap section has a substantially circular cross-section; and
the plurality of cap sections is arranged as a toroid about the diameter of the stem portion.

29. The method of claim 28, wherein the stent is made from a material having shape-memory characteristics.

30. The method of claim 29, wherein at least one of the cap portion and the stem portion is coated with a medicinal coating.
